(19) United States
(12) Patent Application Publication
(10) Pub. No.: US 2021/0186274 A1
(43) Pub. Date: Jun. 24, 2021

(54) APPARATUS AND METHOD FOR DISPENSING A TOWEL OR A CLEANING LIQUID

(71) Applicant: WELLGO Gerätetechnik GmbH, Nohfelden (DE)

(72) Inventors: Jürgen MECHENBIER, Ottweiler (DE); Thorsten MECHENBIER, Ottweiler (DE); Michael LENHARD, Friedrichsthal (DE); Nico HASER, Sulzbach (DE)

(21) Appl. No.: 17/129,003

(22) Filed: Dec. 21, 2020

(30) Foreign Application Priority Data
Dec. 23, 2019 (DE) 10 2019 135 750.0

(51) Int. Cl. A47K 10/32; G06K 19/07; G06K 7/10; A47K 5/12

(52) U.S. Cl. CPC A47K 10/32; G06K 19/0723; A47K 2010/3226; A47K 5/1202; G06K 7/10366

(57) ABSTRACT

An apparatus for dispensing a towel and/or a liquid, which stores information pertaining to an amount of the towel and/or liquid that is arranged in the apparatus and/or dispensed by the apparatus. The apparatus stores the information in a control data memory that is able to be written to only once. The control data memory is part of a radio chip. The apparatus stores the information pertaining to the dispensed amount in a comparison data memory that is erasable and rewritable. The apparatus automatically compares the information pertaining to the dispensed amount of the towel and/or liquid that is stored in the control data memory and in the comparison data memory with one another. The apparatus prevents the towel and/or liquid from being dispensed, or stores the information pertaining to the amount that is stored in the control data memory in the comparison data memory, if the information of the control data memory and that of the comparison data memory are not consistent with one another.

### CROSS-REFERENCE TO RELATED APPLICATIONS

The present application claims priority of DE 10 2019 135 750.0, filed Dec. 23, 2019, the priority of this application is hereby claimed and this application is incorporated herein by reference.

### BACKGROUND OF THE INVENTION

The invention relates to an apparatus for dispensing a towel, in particular a hand towel, or a cleaning liquid, in particular soap or disinfectant, which is intended to store information pertaining to an amount of the towel or cleaning liquid that is arranged in the apparatus and/or dispensed by means of the apparatus.

The invention further relates to a carrier for the towel or cleaning liquid for arrangement in the apparatus and to a method for dispensing the towel or cleaning liquid.

EP 1 956 954 B1 describes such a dispenser apparatus for dispensing sheet material arranged on a reel that is replaceable in the dispenser apparatus. The dispenser apparatus uses a chip arranged on the reel to store the length of the sheet material dispensed from the reel so that it can be established when there is no more sheet material on the reel.

### SUMMARY OF THE INVENTION

The invention is based on the object of preventing the stored information from being manipulated.

According to the invention, this object is achieved by virtue of the apparatus being intended to store the information such that it is not alterable after storage.

The information, which is preferably stored on a control data memory of a towel or cleaning liquid carrier that should be provided in the apparatus and from which the towel or liquid is dispensed, is not able to be changed, thanks to the invention.

Advantageously, unlike in the case of the known apparatus, the invention can preclude use of the towel or liquid carrier if it has been provided with the towel or cleaning liquid without authorization. In the known apparatus, the information is deposited in an alterable, in particular erasable and rewritable, data memory of the chip that contains not only the length of the dispensed sheet material but also information about the identity of the chip and the quality of the sheet material. In the known apparatus, it is therefore possible to alter the stored information pertaining to the length of the sheet material in order to reuse the chip, e.g. with a different or freshly filled reel.

The aforementioned towel or liquid carrier, on which the control data memory is preferably arranged, can be a reel, e.g. made from cardboard or plastic, onto which the towel is rolled, or a liquid container in which the cleaning liquid is arranged, in particular a bottle, a syringe plunger or a drum.

The towel to be dispensed can be for example a hand towel, in particular a paper or cloth hand towel. The cleaning liquid is preferably soap or disinfectant.

The apparatus is expediently configured to hold the towel or liquid carrier mechanically. To this end, it is preferably provided with appropriate holding means.

Preferably, the apparatus is intended to detachably anchor the towel or liquid carrier.

In one embodiment of the invention, the apparatus comprises a dosing dispenser, preferably a soap and/or disinfectant dispenser, or a hand towel dispenser, preferably a paper hand towel or cloth hand towel dispenser.

The towel or liquid carrier is expediently configured for, preferably detachably anchored, arrangement in the apparatus. To this end, it can be provided with holding means that allow the arrangement, in particular anchoring, in the apparatus.

The apparatus is expediently configured to store the information via a connection for data transmission when the towel or liquid carrier is arranged in the apparatus. The data transmission connection can be made by radio, e.g. by means of near field communication (NFC for short), and/or via a data line. If the information is provided via a data line, data line sections of the apparatus and of the towel or liquid carrier are preferably connected to one another when the towel or liquid carrier is arranged in the apparatus, for example by means of plugs or the like.

In order to perform the storage process, the apparatus is expediently provided with a computer, preferably a processor.

Expediently, the apparatus has a device for measuring the dispensed amount of the towel or cleaning liquid. If the towel is arranged on a reel, the dispensed amount can be ascertained e.g. from the rotation of the reel. If the towel to be dispensed is provided as a multiplicity of single sheets, the amount can be measured by counting the single sheets dispensed. If the cleaning liquid is dispensed by means of a pump, the amount can be determined from the dispensed pump volume, in particular on the basis of the pumping operations performed. Alternatively, the mass of the towel or the cleaning liquid arranged on or in the towel or liquid carrier could also be measured and the dispensed amount could be ascertained on the basis of the change of mass.

In one refinement of the invention, the, preferably electronic, control data memory is able to be written to only once. The control data memory could be a one-time programmable module, for example.

In one embodiment of the invention, the control data memory is written to progressively as the towel or cleaning liquid is dispensed. Preferably, data are written to the control data memory each time a prescribed amount of the towel or cleaning liquid has been dispensed, for example after gradual dispensing of a respective prescribed length of towel or mass of the towel or of a prescribed volume or a prescribed mass of the cleaning liquid. The amount is preferably determined by means of the aforementioned measuring device.

One option is for the respectively dispensed amount or the respectively remaining amount to be progressively written to the control data memory, particularly preferably each time the respectively prescribed amount has been dispensed.

In one preferred embodiment of the invention, a data unit, for example a bit of a binary system, of the control data memory is written to, for example with the binary digit “1”, each time the prescribed amount has been dispensed. If the number of data units written to is linked to the amount of dispensed towel or cleaning liquid, this information from the control data memory can be used to determine the amount dispensed from the towel or liquid carrier or remaining on the towel or liquid carrier. Preferably, the data storage on the control data memory is provided such that the control data memory is written to completely if the towel or cleaning liquid is dispensed from the towel or liquid carrier completely or at least almost completely. If the control data memory has been written to fully, the apparatus blocks any further dispensing from the towel or liquid carrier.

It goes without saying that if the towel or liquid carrier has been emptied completely, the control data memory does not necessarily have to be written to completely, but rather a different proportion of memory space written to could alternatively also be provided, e.g. 80 or 90%.

In one preferred refinement of the invention, the apparatus is intended to further store the information pertaining to the dispensed amount in a comparison data memory, which is preferably erasable and rewritable.

The information can be stored in the comparison data memory in the same or a different manner as/from in the control data memory. Storage in a different manner can be advantageous if the comparison data memory has a larger capacity for storing the information than the control data memory and therefore the dispensed or remaining amount can be stored in smaller steps than on the control data memory.

In a further refinement of the invention, the apparatus is intended to automatically compare, preferably by means of the aforementioned or a different processor, the information pertaining to the dispensed amount that is stored in the control data memory and in the comparison data memory with one another. If the same information is deposited in the control data memory and in the comparison data memory, the information can be compared with one another directly.

If, as according to one embodiment of the invention, there is provision for the dispensed amount or the respective remaining amount, e.g. as an amount indication, to be progressively written to the comparison data memory and for the control data memory to have a data unit written to it each time the prescribed amount has been dispensed, as explained above, the apparatus can be intended to perform an algorithm in order to check whether the information in the control data memory and that in the comparison data memory are consistent with one another, i.e. contain matching information about the dispensing.

If the comparison reveals that the information in the control data memory is not consistent with that in the comparison data memory, it follows that the information in the comparison data memory has been manipulated.

The apparatus is expediently intended to prevent the towel or cleaning liquid from being dispensed by means of the apparatus, or to write information pertaining to the amount of the towel or cleaning liquid that is consistent with the information stored in the control data memory to the comparison data memory, if the amount-dispensed information of the control data memory and that of the comparison data memory are not consistent.

In the first case mentioned, no further towel or cleaning liquid can be dispensed using the control data memory. In the second case mentioned, only the amount of the towel or cleaning liquid for which storage space has not yet been used on the control data memory can be dispensed using the control data memory.

In one embodiment of the invention, the control data memory and if need be the comparison data memory is/are part of an RFID transponder. The apparatus expediently comprises an RFID reader and/or writer to read and/or write to the RFID transponder. Said apparatus is preferably configured to write the information to the control data memory and if need be the comparison data memory.

In one refinement of the invention, the apparatus is intended to read from the comparison data memory and/or the control data memory an identifier on the basis of which it is checkable whether the towel or liquid carrier is authorized for use in the apparatus.

Optionally, the identifier can also contain information about the towel or cleaning liquid, for example about the type, quality and/or amount of towel or cleaning liquid arranged on or in the towel or liquid carrier.

Expediently, the identifier and if need be also the stored information is/are encrypted by means of an identification of the RFID transponder (“ID”).

The various features of novelty which characterize the invention are pointed out with particularity in the claims annexed to and forming a part of the disclosure. For a better understanding of the invention, its operating advantages, specific objects attained by its use, reference should be had to the drawings and descriptive matter in which there are illustrated and described preferred embodiments of the invention.

### BRIEF DESCRIPTION OF THE DRAWING

In the drawing:

FIG. 1 shows an apparatus according to the invention in different views,

FIG. 2 schematically shows the design of components of the apparatus according to the invention shown in FIG. 1,

FIG. 3 shows a further apparatus according to the invention in different views, and

FIG. 4 schematically shows the design of components of the apparatus according to the invention shown in FIG. 3.

### DETAILED DESCRIPTION OF THE INVENTION

In an exemplary embodiment that is shown in FIG. 1, the apparatus 1 according to the invention is in the form of a paper hand towel dispenser. FIG. 1a shows a housing 9 of the apparatus 1, in which there is provision for a dispensing opening 10 through which a paper hand towel is dispensed outward. As FIGS. 1b and c show, a carrying reel on which the paper hand towel 6 is rolled up is arranged in the housing 9 on a reel holder 8 as hand towel carrier 5. To dispense the paper hand towel 6, there is provision for a drive 11 by means of which the paper hand towel 6 together with the hand towel carrier 5 can be set in rotation.

FIG. 1d uses a section through the reel holder 8 and the hand towel carrier 5 to show that an RFID reader/writer 7 is arranged in the reel holder 8. An RFID transponder 3 that can be read and written to using the RFID reader/writer 7 sits in the carrying reel 5.

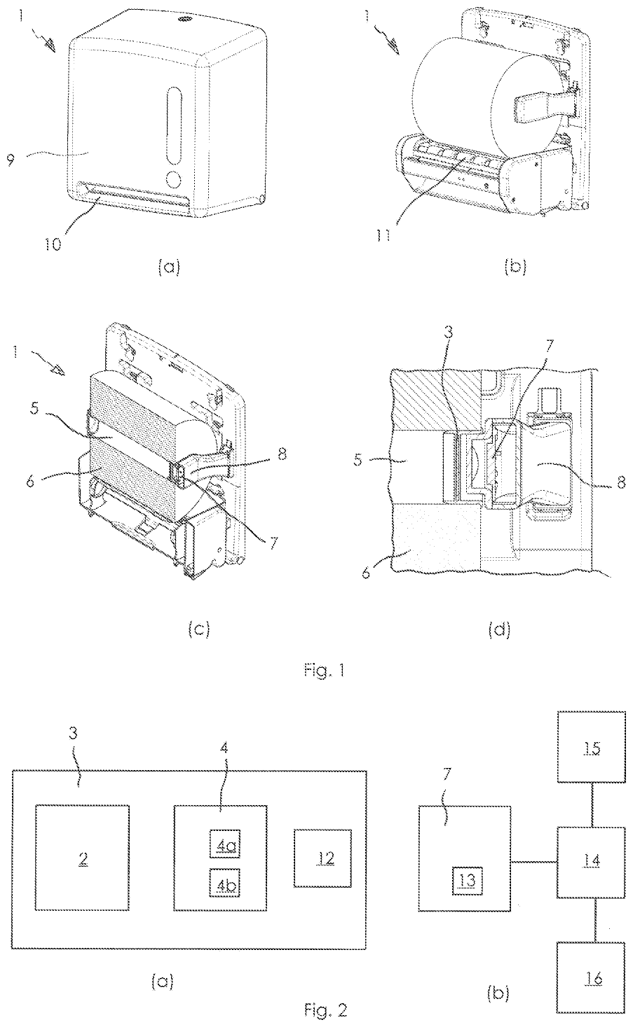

A measuring device 15, shown merely schematically in FIG. 2b in this case, that can be used to measure the number of revolutions of the hand towel carrier 5 in the apparatus 1 is further formed in the reel holder 8. From the number of revolutions, the length of dispensed paper hand towel 6 can be determined by means of a computer 14 of the apparatus 1 and stored in a data memory 16 of the apparatus 1.

FIG. 2a schematically shows that the RFID transponder 3 has a control data memory 2, which can be written to just a single time and cannot be erased or written to again. The control data memory 2 can be a read-only memory, in particular an electronic one-time programmable module.

The RFID transponder 3 furthermore has a further, erasable and rewritable, data memory 4. A data memory area 4a of the data memory 4 has an identifier of the RFID transponder 3 deposited in it, from which the computer 14 of the apparatus 1 can read, by means of the RFID reader/writer 7, whether the hand towel carrier 5 is authorized for use in the apparatus 1.

The data transmission between the RFID reader/writer 7 and the RFID transponder 3 is effected by radio via an antenna 12 of the RFID transponder 3 and an antenna 13 of the RFID reader/writer 7.

In the example explained below, the paper hand towel 6 in a length of 320 m is provided on the hand towel carrier 5 and a 32-bit memory is provided as control data memory 2.

When the paper hand towel 6 is dispensed from the apparatus 1, the measuring device 15 is used to determine the length of the dispensed paper hand towel 6, e.g. from revolutions of the hand towel carrier 5 in the apparatus. Whenever 10 m of the paper hand towel 6 has been dispensed, the RFID reader/writer 7 is used to write the digit “1” to a bit of the control data memory 2 each time. The dispensed paper hand towel length can be ascertained from the control data memory 2 with an accuracy of 10 m, e.g. by means of the computer 14. In particular, the paper hand towel 6 has been dispensed from the hand towel carrier 5 completely when the 32 bits of the control data memory 2 have been written to completely.

The computer 14 of the apparatus 1 is programmed such that dispensing of the paper hand towel 6 is permitted only if the control data memory 2 has not been written to completely.

Since the apparatus 1 repeatedly writes to the control data memory 2 following gradual dispensing of 10 m of the paper hand towel 6 each time, the hand towel carrier 5 provided with the RFID transponder 3 can consequently be used to dispense just the aforementioned 320 m of the paper hand towel 6. Even if the hand towel carrier 5 were to be refilled with new paper hand towel after the paper hand towel 6 has been dispensed, it would not be possible for more than the 320 m to be dispensed from the hand towel carrier 5. Advantageously, this prevents paper hand towel placed onto the hand towel carrier 5 without authorization from being able to be dispensed by means of the apparatus 1.

In a further exemplary embodiment, in addition to the storage in the control data memory 2, the information about the length of the dispensed paper hand towel 6 is stored in a comparison data memory 4b, which is a data memory area of the further, erasable and rewritable, data memory 4. In order to be able to determine the length of the dispensed paper hand towel 6 more accurately, appropriate information is stored in the comparison data memory 4b each time 2 m of the paper hand towel 6 has been dispensed.

The computer 14 of the apparatus 1 is intended to use an algorithm to compare the information from the control data memory 2 with that of the comparison data memory 4b. Should the length information determinable therefrom be consistent with one another, i.e. the length information from the comparison data memory 4b that has been created in 2 m steps is within the length information from the control data memory 2, which is provided in 10 m steps, paper hand towel dispensing by means of the apparatus 1 is enabled. Otherwise, i.e. if the information in the comparison data memory 4b has been manipulated, dispensing is blocked.

Alternatively, there can be provision for length information consistent with the information from the control data memory 2 to be written to the comparison data memory 4b if the information from the control data memory 2 is consistent with that from the comparison data memory 4b. The hand towel carrier 5 can then dispense as much paper hand towel 6 as there is provision for on the basis of the information from the control data memory 2.

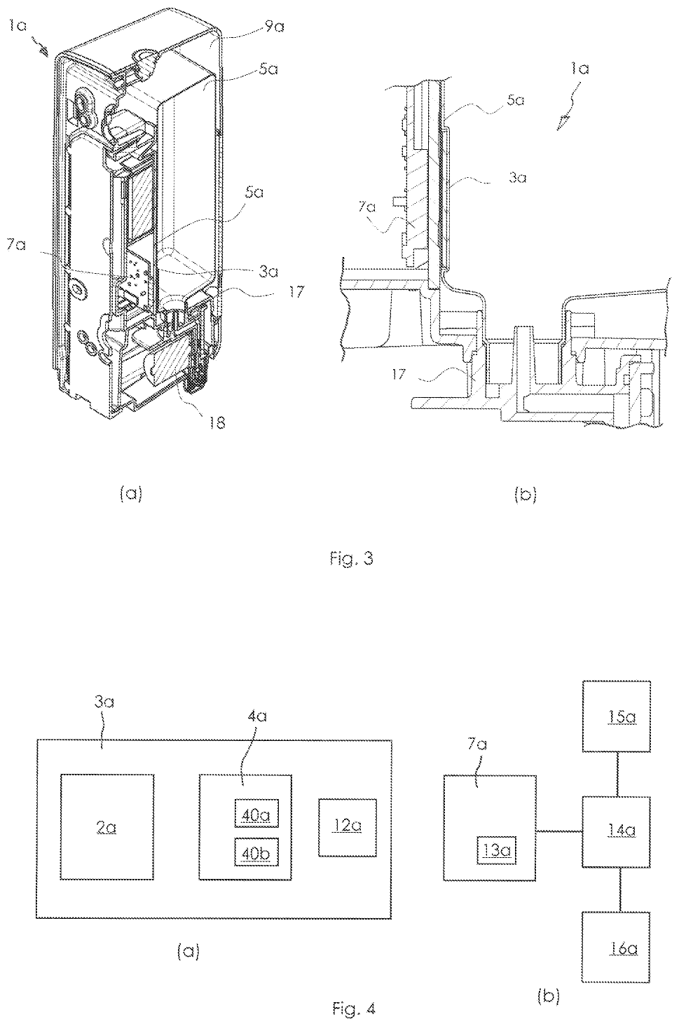

FIG. 3 shows a liquid dispenser, intended for dispensing soap or disinfectant, as a further apparatus 1a according to the invention. The apparatus has a housing 9a that has an area for receiving a liquid container 5a. The liquid container 5a is provided with an outlet 19 through which the liquid can be dispensed. To dispense the liquid in a controlled manner, there is provision for a pump, e.g. a geared pump, hose pump or piston pump, that can be operated by means of a pump motor 18.

Arranged inside the housing 9a is an RFID reader/writer 7a, beside which an RFID transponder 3a of the liquid container 5a is arranged when the liquid container 5a sits in the housing.

FIG. 4a schematically shows that the RFID transponder 3a has a control data memory 2a, e.g. an electronic one-time programmable module, which can be written to just a single time and cannot be erased or written to again.

The RFID transponder 3a furthermore has a further, erasable and rewritable, data memory 40. A data memory area 40a of the data memory 40 has an identifier of the RFID transponder 3a deposited in it, from which the computer 14a of the apparatus 1a can read, by means of the RFID reader/writer 7a, whether the liquid container 5a is authorized for use in the apparatus 1a. The data transmission between the RFID reader/writer 7a and the RFID transponder 3a is effected by radio via an antenna 12a of the RFID transponder 3a and an antenna 13a of the RFID reader/writer 7a.

The apparatus 1a has a computer 14a and a data memory 16a and is also configured by means of a pump meter 15a, shown only in FIG. 4b, to count the pumping operations. From the number of pumping operations, the volume of dispensed liquid can be determined by means of the computer 14a.

In the example explained below, the liquid container contains 640 ml of soap 6a and the control data memory 2a provided is a 32-bit memory. The pump 17 is configured such that 20 ml of the soap 6a is dispensed for each pumping operation.

When the soap 6a is dispensed from the apparatus 1a, the volume of the dispensed soap 6a is determined by means of the apparatus 14a and the pump meter, as explained above. Whenever a pumping operation has been performed and the 20 ml of the soap 6a has been dispensed, the RFID reader/writer 7 is used to write the digit “1” to a bit of the control data memory 2 each time. 640 ml of the soap 6a has been dispensed from the liquid container when the 32 bits of the control data memory 2 have been written to completely.

The computer 14a of the apparatus 1a is programmed such that dispensing of the soap 6a is permitted only if the control data memory 2a has not been written to completely.

Since the apparatus 1a repeatedly writes to the control data memory 2a following gradual dispensing of 20 ml of the soap 6a each time, only the aforementioned 640 ml of the soap 6a can be dispensed using the liquid container 5a provided with the RFID transponder 3a. Even if the liquid container 5a has been refilled when the soap 6a has been dispensed, no more than the 640 ml could be dispensed in total. This prevents the liquid container 5a from being able to be refilled without authorization and the soap 6a put into the liquid container 5a without authorization from being able to be dispensed by means of the apparatus 1a.

In a further exemplary embodiment, in addition to the storage in the control data memory 2a, the information about the volume of the dispensed soap 6a is stored in a comparison data memory 40b, which is a data memory area of the further, erasable and rewritable, data memory 40. In order to be able to determine the volume more accurately, appropriate information is stored in the comparison data memory 40b each time 5 ml of the soap 6a has been dispensed.

The computer 14a is intended to use an algorithm to compare the information from the control data memory 2a with that of the comparison data memory 40b. Should the length information determinable therefrom be consistent with one another, i.e. the length information from the comparison data memory 40b that has been created in 5 ml steps is within the volume information from the control data memory 2a, which is provided in 20 ml steps, soap dispensing by means of the apparatus 1 is enabled. Otherwise, i.e. if the information in the comparison data memory 40b has been manipulated, dispensing is blocked.

Alternatively, there can be provision for volume information consistent with the information from the control data memory 2a to be written to the comparison data memory 40b if the information from the control data memory 2a is consistent with that from the comparison data memory 40b. The liquid container 5a can then dispense as much soap 6a as there is provision for on the basis of the information from the control data memory 2a.

It goes without saying that the liquid container 5a could also be filled with disinfectant instead of the aforementioned soap 6a.

While specific embodiments of the invention have been shown and described in detail to illustrate the inventive principles, it will be understood that the invention may be embodied otherwise without departing from such principles.

We claim:

1. An apparatus for dispensing a towel or a cleaning liquid, comprising an information storage that retains information pertaining to an amount of the towel or cleaning liquid that is arranged in the apparatus and/or dispensed by the apparatus, wherein the storage is configured to store the information so that the information is not alterable after storage.

2. The apparatus according to claim 1, wherein the information storage includes a control data memory that is able to be written to only once.

3. The apparatus according to claim 2, wherein the control data memory is electronic.

4. The apparatus according to claim 2, wherein the control data memory is part of a radio chip.

5. The apparatus according to claim 4, wherein the control data memory is an RFID transponder.

6. The apparatus according to claim 2, wherein the control data memory is a read-only memory.

7. The apparatus according to claim 6, wherein the control data memory is a one-time programmable module.

8. The apparatus according to claim 2, wherein the information storage includes a comparison data memory.

9. The apparatus according to claim 8, wherein the comparison data memory is erasable and rewritable.

10. The apparatus according to claim 8, wherein the information storage is configured to automatically compare the information pertaining to the dispensed amount that is stored in the control data memory and in the comparison data memory with one another.

11. The apparatus according to claim 10, wherein the apparatus is configured to prevent the towel or cleaning liquid from being dispensed, or to store the information pertaining to the amount that is stored in the control data memory in the comparison data memory, if the amount-dispensed information of the control data memory and of the comparison data memory are not consistent with one another.

12. The apparatus according to claim 10, further comprising a towel or liquid carrier, wherein the towel or liquid carrier is provided with the control data memory and if need be the comparison data memory.

13. The apparatus according to claim 1, further comprising a dosing dispenser or a hand towel dispenser.

14. A towel or liquid carrier for arrangement in an apparatus for dispensing a towel or a cleaning liquid, wherein the towel or liquid carrier is provided with a control data memory for storing information pertaining to an amount of the towel or cleaning liquid dispensed by the towel or liquid carrier, and the control data memory is configured so that the stored information is not alterable after it is stored.

15. The towel or liquid carrier according to claim 14, further comprising a comparison data memory that is alterable.

16. The towel or liquid carrier according to claim 15, wherein the towel or liquid carrier is configured so that the information is storable in the control data memory and if need be the comparison data memory by the apparatus.

17. A method for dispensing a towel or a cleaning liquid, comprising the steps of: providing an apparatus for holding and dispensing the towel or liquid; and storing information pertaining to an amount of the towel or liquid dispensed by the apparatus, wherein the information is stored so that the information is not alterable after the information is stored.

18. The method according to claim 17, wherein the information is stored in an electronic control data memory that is able to be written to only once and in an erasable and rewritable comparison data memory

19. The method according to claim 18, including automatically comparing the information stored in the control data memory and in the comparison data memory with one another.

20. The method according to claim 19, including preventing the towel or liquid from being dispensed, or storing the information pertaining to the amount that is stored in the control data memory in the comparison data memory, if the amount-dispensed information of the control data memory and the amount dispensed information of the comparison data memory are not consistent.
